(19) United States
(12) Patent Application Publication
(10) Pub. No.: US 2011/0195214 A1
(43) Pub. Date: Aug. 11, 2011

(54) SUPERABSORBENT BI-COMPONENT FIBER

(76) Inventors: Kay Bernhard, Darmstadt (DE); Jörg Dahringer, Darmstadt (DE); Michael Klanert, Schwabmunchen (DE); Werner Stefani, Grossaitingen (DE)

(21) Appl. No.: 13/122,760

(22) PCT Filed: Oct. 6, 2009
(86) PCT No.: PCT/EP09/07160

§ 371 Date: Apr. 6, 2011

(30) Foreign Application Priority Data
Oct. 11, 2008 (DE) 10-2008-051-430.6

(51) Int. Cl. B32B 5/02; D02G 3/36; D01D 5/22; D01D 5/34; D01D 5/32; D04H 13/00; B32B 1/02; A61L 15/22

(52) U.S. Cl. 428/36.1; 428/372; 264/168; 264/148; 428/221; 442/334; 604/370

(57) ABSTRACT

A superabsorbent bi-component fiber, wherein component A is at least one thermoplastic polymer and component B is a compound selected from at least one thermoplastic base polymer and at least one superabsorbent polymer (SAP), and also a method for production thereof are described. The melting point of the thermoplastic contained in component A is at least 20° C. higher than the melting point of the thermoplastic contained in component B, the average grain size of the SAP is 0.5 to 10 μm and the compound has an SAP-fraction of 0.5 to 40 wt %. The bi-component fiber can be used to produce superabsorbent textile fabrics which are used in particular in the field of hygiene and medicine.

The invention relates to a superabsorbent bi-component fiber, a method for the production thereof, superabsorbent textile surface structures produced therefrom and its use in particular in the hygiene industry and medicine.

Bi-component fibers enriched with a superabsorbent polymer (SAP) and their use as non-woven fabrics are described in DE-A-10232078. They are core-sheath fibers in which the core and the base material forming the sheath are made of a thermoplastic polymer, in particular a polyolefin. The core is free from SAP; the sheath contains a fibrous compound of the thermoplastic polymer with SAP and forms 10 to 50% of the cross-sectional area of the bi-component filaments. The compound forming the sheath contains 5 to 50% by weight of SAP which has an average particle size of 1 to 50 μm. The production of the bi-component filaments is performed by co-extrusion of the two previously mentioned polymer mixtures. A spunbonded non-woven fabric can also be obtained in one process step by means of an appropriate melt-spinning process.

However, the previously described bi-component fibers still need to be improved in terms of spinning behavior, die lives and mechanical stability.

Thus, the object is to provide a superabsorbent bi-component fiber which has a good mechanical stability and at the same time a good water absorption capacity.

The above-mentioned object is achieved by the superabsorbent bi-component fiber in which the component A contains at least one thermoplastic polymer and the component B contains a compound of at least one thermoplastic base polymer and at least one superabsorbent polymer (SAP), characterized in that
the melting point of the thermoplastic comprised by the component A is by at least 20° C. higher than the melting point of the thermoplastic comprised by the component B,
the average particle size of the SAP is 0.5 to 10 μm, and
the compound has an SAP content of 0.5 to 40% by weight.

The term “bi-component fibers” is to be understood to mean bi-component or multi-component fibers which have a side-by-side structure or a core-sheath structure. According to the invention, bi-component fibers having a core-sheath structure are preferred, the component A being comprised by the core and the component B being comprised by the sheath.

Thermoplastic polymers with a high melting point (mp≧100° C.) which are suitable for the production of fibers are preferably used for the component A.

Suitable polymer materials include, amongst others, e.g., polyamides such as, e.g., polyhexamethylene adipinamide, polycaprolactam, aromatic or partially aromatic polyamides (“aramids”), aliphatic polyamides such as, e.g., nylon, partially aromatic or fully aromatic polyesters, polyphenylene sulfide (PPS), polymers with ether and keto groups such as, e.g., polyether ketones (PEK) and polyether ether ketone (PEEK), polyolefins such as, e.g., polyethylene or polypropylene.

Preference is given to melt-spinnable polyesters.

The polyester material can, in principle, be any known type suitable for fiber production. Melt-spinnable polyesters predominantly consist of building blocks which are derived from aromatic dicarboxylic acids and aliphatic diols. Common aromatic dicarboxylic acid-building blocks are the divalent radicals of benzenedicarboxylic acids, in particular of terephthalic acid and isophthalic acid; common diols have 2 to 4 C atoms, ethylene glycol and/or propane-1,3-diol being particularly suitable.

It is particularly advantageous if the component A of the bi-component fiber consists to at least 85 mol % of polyethylene terephthalate (PET) and/or polytrimethylene terephthalate (PTT). The remaining 15 mol % are then formed by dicarboxylic acid moieties and glycol moieties which act as so-called modifiers and allow the person skilled in the art to specifically influence the physical and chemical properties of the produced filaments. Examples of such dicarboxylic acid moieties are radicals of isophthalic acid or aliphatic dicarboxylic acid such as, e.g., glutaric acid, adipic acid, sebacic acid; examples of diol radicals with a modifying action are those of longer-chain diols, e.g., propanediol or butanediol, diethylene or triethylene glycol or, if present in minor amounts, polyglycol having a molecular weight of about 500 to 2000.

Polyesters containing at least 95 mol % of polyethylene terephthalate (PET) are particularly preferred as component A, especially those composed of unmodified PET.

Such polyesters usually have a molecular weight corresponding to an intrinsic viscosity (IV) of 0.4 to 1.4 (dl/g), measured on solutions in dichloroacetic acid at 25° C.

Suitable thermoplastic base polymers for the component B are polyolefins, preferably polyethylene and/or polypropylene, or copolyesters, the melting point of the thermoplastic comprised by the component A being by at least 20° C. higher than the melting point of the thermoplastic comprised by the component B.

The above-mentioned polymers can be employed as homopolymers or copolymers alone and/or in the form of mixtures thereof.

Polyethylene (PE) is preferably used as the base polymer. Customary, in particular commercially available polyethylene grades can be used.

These include in particular fiber-forming linear ethylene polymers such as, e.g., HDPE, LDPE and/or LLDPE. Such ethylene polymers are described in WO 2004/033771.

Cross-linked polymers of acrylic acid (partially neutralized and slightly surface-cross-linked) are referred to as SAP which are able to absorb a multiple of their own weights—up to 1000 times the weight—in fluids (e.g. water or bodily fluids) while forming a gel and can also store these under pressure.

The choice of SAP employed according to the invention is not subject to any limitations. Customary superabsorbers can be used as SAP, such as, e.g., those of the brand FAVOR® (=registered trademark of Evonik), OASIS SAF® (=registered trademark of Technical Absorbents Ltd.), Luquasorb® (=registered trademark of BASF), Aquakeep®, Norsocryl® (=registered trademarks of Arkema) and/or AQUALIC CA® (=registered trademark of Nippon Shokubai).

The SAP employed according to the invention should preferably have sufficient thermal stability with regard to the melt-spinning process.

In the case of the SAP employed according to the invention, this is ground by means of common grinding methods to an average particle size (=D90) of preferably 1 μm to 10 μm, particularly preferably 1 μm to 5 μm. The proportion of SAP comprised therein having a particle size of more than 15 μm must not exceed 1% by weight so that the spinning process is not disrupted by these.

The determination of the average particle size is performed by a laser light scattering method in accordance with ISO 13320-1. For example, a Microtrac S 3500 is a suitable measuring device for the particle size analysis.

The ground superabsorber (SAP) is compounded into the base polymer of component B by means of a mixing extruder, for example. This compound can serve as the masterbatch for the component B or as the only raw material, depending on the filling degree. Generally, the compound has an SAP content of from 0.5 to 40% by weight, preferably from 1 to 35% by weight, particularly preferably from 5 to 30% by weight.

The production of the bi-component fiber according to the invention is performed according to customary methods. Initially, the components A and B (i.e. the compound described above) are provided and spun to bi-component filaments by co-extrusion. For this, customary devices with appropriate dies are used. The exit velocity at the die mouth area is matched with the spinning velocity such that a fiber with the desired titer is formed.

The co-extrusion should preferably be carried out in such a way that the compound (component B) forms 20-80% of the cross-sectional area of the bi-component filaments.

Spinning velocity is to be understood to mean the velocity at which the solidified strands are drawn off. The strands such drawn off can either be fed directly to the drafting or also only be wound or laid down and drafted at a later point in time. The fibers and filaments drafted in a customary manner can then be crimped, set and/or cut to the desired length to staple fibers.

The single titer of the bi-component fibers according to the invention is in its final form between 0.9 and 30 dtex, preferably 0.9 to 13 dtex.

To prevent premature swelling of the SAP, spinning preparations and Avivagen on a non-aqueous basis or systems with negligible swelling capacity (e.g. by addition of salt) are used.

Corresponding superabsorbent textile surface structures which are likewise an object of the invention can be produced from the superabsorbent bi-component fibers according to the invention.

Within the context of this description, the term “textile surface structure” is to be understood in its widest meaning. It can be any structure containing the fibers according to the invention which has been produced according to a surface-forming technology. Examples of such textile surface structures are fabrics, layings, knitting fabrics and knitwear as well as preferably non-woven fabrics.

The non-woven according to the invention can be formed from continuous synthetic fibers or staple fibers. Superabsorbent bi-component staple fibers according to the invention are preferably used for the non-woven. The length of the above-mentioned staple fibers is generally of from 1 to 200 mm, preferably 3 to 120 mm, particularly preferably 3 to 60 mm.

In another embodiment of the invention, the textile surface, in particular the non-woven fabric, can additionally be consolidated by a hot-melt binder. For this, carrier and hot-melt fibers are additionally added which can be derived from any thermoplastic, fiber-forming polymers. Moreover, carrier fibers can also be derived from non-melting, fiber-forming polymers. Such spunbonded non-woven fabrics consolidated by a hot-melt binder are described, for example, in EP-A 0446822 and EP-A 0590629.

Examples of polymers from which the carrier fibers can be derived are polyacrylonitrile, polyolefins, such as polyethylene or polypropylene, primarily aliphatic polyamides, such as nylon 6.6, primarily aromatic polyamides (aramids), such as poly-(p-phenylene terephthalate) or copolymers containing a content of aromatic m-diamine moieties to improve the solubility, or poly-(m-phenylene isophthalate), primarily aromatic polyesters, such as poly-(p-hydroxybenzoate), or preferably primarily aliphatic polyesters, such as polyethylene terephthalate.

The proportion of the additional carrier and hot-melt fibers to each other can be chosen within wide limits, it being necessary in this connection to chose the proportion of hot-melt fibers that high that the non-woven achieves a strength sufficient for the desired application by bonding the carrier fibers with the hot-melt fibers. The proportion of the hot-melt derived from the hot-melt fiber in the non-woven is usually less than 50% by weight, based upon the weight of the non-woven.

Modified polyesters with a melting point decreased by 10 to 50° C., preferably 30 to 50° C. in comparison to the non-woven raw material particularly come into consideration as hot-melts. Examples of such a hot-melt are polypropylene, polyethylene, polybutylene terephthalate or polyethylene terephthalate modified by condensing longer-chain diols and/or isophthalic acid or aliphatic dicarboxylic acid into the polyethylene terephthalate.

The textile surface structures produced from the fibers according to the invention can also be subjected to a mechanical and/or chemical consolidation. The consolidation can be carried out by means of known methods. Without limiting the possible methods with this, mechanical methods, such as needling, in particular hydrodynamic consolidation with a fluid which does not lead to swelling of the fibers, as well as chemical and/or thermoplastic methods are suitable.

The thermal consolidation of the textile surface structures is generally carried out via the hot-melt bonding capacity of the bi-component fibers according to the invention comprised therein. Furthermore, the textile surface structure can additionally also be consolidated by chemical binders, in particular those based on acrylates or styrenes.

The textile surface structure can be formed by one or several layers, at least one layer comprising the fibers according to the invention.

Non-wovens produced from the bi-component fibers according to the invention have the advantage that they combine the mechanical stability and the hot-melt bonding capacity of a bi-component staple fiber with the water absorption capacity of superabsorbent polymers.

Due to their high SAP content and the ability to absorb fluids associated therewith, the non-wovens produced from the bi-component fibers according to the invention can be employed advantageously for applications in the hygiene industry and medicine. Moreover, they can be employed for special packaging for foodstuff, leakage protection for packaging for fluids and in technical areas in which moisture has to be avoided.

The use of such non-wovens is of particular significance for hygiene products, such as diapers, incontinence products, sanitary napkins etc. With these products for daily use, it happens frequently that these are disposed off with the waste water which can lead to the blockage of the sewage system.

By choosing suitable filling degrees (SAP content in the base polymer of component B) of from 10 to 35% by weight, preferably 15 to 30%, in particular 20 to 30% by weight, it can be achieved that the non-woven structure of the non-woven fabric according to the invention is broken up by forcing the glue dots open when the SAP swells. Through this, the product is largely broken down into its individual fibers, having the advantage that no blockage of the sewage system occurs even when the product is disposed of into the latter.

According to the invention, bi-component fibers with a filling degree of 10 to 35% by weight in which the compound (component B) forms 20-80% of the cross-sectional area of the bi-component filaments, an average particle size (D90) of the SAP of from 1 to 10 μm and a length of the staple fibers of from 3 to 60 mm are preferred.

The invention is clarified by the following example without limiting the scope of the invention on this example.

### EXAMPLE FOR THE PRODUCTION OF A CORE-SHEATH BI-COMPONENT FIBER

Customary superabsorber FAVOR 4000 from Evonik is ground in a fluidized-bed opposed-jet mill to a particle size of d90 <10 μm, determined by laser light scattering with a Microtrac S 3500 measuring device according to ISO 13320-1. The ground superabsorber is compounded into LLDPE as the sheath polymer with 30% by weight by means of mixing extruder.

100 kg of standard PET (IV=0.65+/−0.05 dl/g) as the raw material for the core component is spun together with the SAP-loaded LLDPE sheath compound in a customary manner to bi-component staple fibers. To prevent premature swelling of the SAP, an ester oil is used a spinning preparation. The spun product is placed into spinning cans until it is processed further.

A dimensional stable fiber is produced from the spun product on the fiber conveyor line by drafting, crimping and thermal treatment. To this end, the spun product is gathered as a fiber cable via a grate and drawn in by a first septet consisting of seven rotating rolls, tempered on a second septet and again prepared with the ester oil.

The drafting is performed on the 6^{th} or 7^{th} roll of this septet or between this septet and another septet running faster by the factor of the drafting. Subsequently, the fiber is crimped in a crimping chamber, set or dried in an oven at 100° C. and cut to a length of 6 mm.

1. A superabsorbent bi-component fiber comprising a component A containing at least one thermoplastic polymer, and a component B containing a compound of at least one thermoplastic base polymer and at least one superabsorbent polymer (SAP), characterized in that
a melting point of the thermoplastic comprised by the component A is at least 20° C. higher than a melting point of the thermoplastic comprised by the component B,
an average particle size of the SAP is 0.5 to 10 μm, and
the fiber has an SAP content of 0.5 to 40% by weight.

2. The superabsorbent bi-component fiber according to claim 1, characterized in that the fiber has a core-sheath structure in which the component A comprises the core and the component B comprises the sheath.

3. The superabsorbent bi-component fiber according to claim 1, characterized in that the average particle size of the SAP is 1 μm to 10 μm, in particular.

4. The superabsorbent bi-component fiber according to claim 1, characterized in that the component B has an SAP content of from 1 to 35% by weight.

5. The superabsorbent bi-component fiber according to claim 1 to 4, characterized in that the component A is made of melt-spinnable polyester and the base polymer of the component B is made of polyethylene.

6. The superabsorbent bi-component fiber according to claim 5, characterized in that the component A is made of polyethylene terephthalate.

7. A method for the production of the superabsorbent bi-component fiber comprising the steps of: providing a component A and a component B, spinning the bi-component filaments by co-extrusion, drafting the bi-component fibers, crimping the drafted bi-component fibers, setting and/or cutting the bi-component fibers to a desired length.

8. A textile surface structure containing the superabsorbent bi-component fiber according to claim 1.

9. The textile surface structure according to claim 8, characterized in that the structure is a non-woven.

10. A hygiene product including the textile surface structure according to claim 8.

11. The hygiene product according to claim 10 being a diaper.

12. The hygiene product according to claim 10 being a sanitary napkins or panty liners.

13. A special packaging for foodstuff including the textile surface structure according to claim 8.

14. A packaging for fluids including the textile surface structure according to claim 8.
